SEQUENCE LISTING (10 sequences)

SEQ ID NO: 1 - DNA, 351 bp, synthetic construct
  misc_feature 1..351: note = Synthetic
  source 1..351: mol_type = other DNA
  CDS 1..351
caggtgcagctggtggagtctgggggaggcgttgtgcagcctggaaggtccctgagactctcctgtgcagcctctggattcaccttcagtagctatgacatgtcttgggttcgccaggctccgggcaagggtctggagtgggtcgcaaaagttagtagtggtggtggtagcacctactatttagacactgtgcagggccgattcaccatctccagagacaatagtaagaacaccctatacctgcaaatgaactctctgagagccgaggacacagccgtgtattactgtgcaagacataactacggcagttttgcttactggggccaagggactacagtgactgtttctagt

SEQ ID NO: 2 - protein, 117 aa, synthetic construct
  REGION 1..117: note = Synthetic Construct
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHNYGSFAYWGQGTTVTVSS

SEQ ID NO: 3 - DNA, 321 bp, synthetic construct
  misc_feature 1..321: note = Synthetic
  source 1..321: mol_type = other DNA
  CDS 1..321
gagattgtgctaactcagtctccagccaccctgtctctcagcccaggagaaagggcgactctttcctgccaggccagccaaagtattagcaaccacctacactggtatcaacaaaggcctggtcaagccccaaggcttctcatcaagtatcgttcccagtccatctctgggatccccgccaggttcagtggcagtggatcagggacagatttcaccctcactatctccagtctggagcctgaagattttgcagtctattactgtcaacagagtggcagctggcctcacacgttcggaggggggaccaaggtggaaattaag

SEQ ID NO: 4 - protein, 107 aa, synthetic construct
  REGION 1..107: note = Synthetic Construct
EIVLTQSPATLSLSPGERATLSCQASQSISNHLHWYQQRPGQAPRLLIKYRSQSISGIPARFSGSGSGTDFTLTISSLEPEDFAVYYCQQSGSWPHTFGGGTKVEIK

SEQ ID NO: 5 - DNA, 351 bp, synthetic construct
  misc_feature 1..351: note = Synthetic
  source 1..351: mol_type = other DNA
  CDS 1..351
gaagtgcagctggtggagtctgggggaggcttagtgaagcctggaaggtccctgagactctcctgtgcagcctctggattcgctttcagtagctatgacatgtcttgggttcgccagattccggagaagaggctggagtgggtcgcaaaagttagtagtggtggtggtagcacctactatttagacactgtgcagggccgattcaccatctccagagacaatgccaagaacaccctatacctgcaaatgagcagtctgaactctgaggacacagccatgtattactgtgcaagacataactacggcagttttgcttactggggccaagggactctggtcactgtctctgca

SEQ ID NO: 6 - protein, 117 aa, synthetic construct
  REGION 1..117: note = Synthetic Construct
EVQLVESGGGLVKPGRSLRLSCAASGFAFSSYDMSWVRQIPEKRLEWVAKVSSGGGSTYYLDTVQGRFTISRDNAKNTLYLQMSSLNSEDTAMYYCARHNYGSFAYWGQGTLVTVSA

SEQ ID NO: 7 - DNA, 321 bp, synthetic construct
  misc_feature 1..321: note = Synthetic
  source 1..321: mol_type = other DNA
  CDS 1..321
gatattgtgctaactcagtctccagccaccctgtctgtgacaccaggagatagcgtcagtctttcctgccaggccagccaaagtattagcaaccacctacactggtatcaacaaaaatcacatgagtctccaaggcttctcatcaagtatcgttcccagtccatctctgggatcccctccaggttcagtggcagtggatcagggacagatttcgctctcagtatcaacagtgtggagactgaagattttggaatgtatttctgtcaacagagtggcagctggcctcacacgttcggaggggggaccaagctggaaattaag

SEQ ID NO: 8 - protein, 107 aa, synthetic construct
  REGION 1..107: note = Synthetic Construct
DIVLTQSPATLSVTPGDSVSLSCQASQSISNHLHWYQQKSHESPRLLIKYRSQSISGIPSRFSGSGSGTDFALSINSVETEDFGMYFCQQSGSWPHTFGGGTKLEIK

SEQ ID NO: 9 - DNA, 321 bp, synthetic construct
  misc_feature 1..321: note = Synthetic
  source 1..321: mol_type = other DNA
  CDS 1..321
gagattgtgctaactcagtctccagccaccctgtctctcagcccaggagaaagggcgactctttcctgccaggccagccaaagtattagcaaccacctacactggtatcaacaaaggcctggtcaagccccaaggcttctcatcmkktatcgttcccagtccatctctgggatccccgccaggttcagtggcagtggatcagggacagatttcaccctcactatctccagtctggagcctgaagattttgcagtctattactgtcaacagagtggcagctggcctcacacgttcggaggggggaccaaggtggaaattaag

SEQ ID NO: 10 - protein, 107 aa, synthetic construct
  VARIANT 49: note = misc_feature - The 'Xaa' at location 49 stands for Arg or Met.
  REGION 1..107: note = Synthetic Construct
EIVLTQSPATLSLSPGERATLSCQASQSISNHLHWYQQRPGQAPRLLIXYRSQSISGIPARFSGSGSGTDFTLTISSLEPEDFAVYYCQQSGSWPHTFGGGTKVEIK

(19) United States
(12) Patent Application Publication
(10) Pub. No.: US 2026/0159592 A1
(43) Pub. Date: Jun. 11, 2026

(54) COMPOSITIONS AND METHODS FOR TARGETING AND KILLING ALPHA-V BETA-3 -POSITIVE CANCER STEM CELLS (CSCS) AND TREATING DRUG RESISTANT AND METASTATIC CANCERS

(71) Applicant: The Regents of the University of California, Oakland, CA (US)

(72) Inventors: David Cheresh, Encinitas, CA (US); Hiromi Wettersten, San Diego, CA (US)

(21) Appl. No.: 19/304,416

(22) Filed: Aug. 19, 2025

Related U.S. Application Data
(63) Continuation of application No. 16/945,057, filed on Jul. 31, 2020, now abandoned.
(60) Provisional application No. 62/882,296, filed on Aug. 2, 2019.

(51) Int. Cl. C07K 16/28; A61K 9/00; A61K 39/00; A61K 45/06

(52) U.S. Cl. CPC C07K 16/2848; A61K 9/0019; A61K 2039/505; A61K 45/06; C07K 2317/24; C07K 2317/52

(57) ABSTRACT

In alternative embodiments, provided are compositions and methods for treating or ameliorating an advanced cancer such as a drug resistant or metastatic cancer which express αvβ3 polypeptides on their cell surfaces, or for killing Cancer Stem Cells (CSCs) which express αvβ3 polypeptides on their cell surfaces, by using human or humanized antibodies capable of specifically binding cell surface-expressed αvβ3 (avb3) polypeptides whose Fc region has a selective affinity to human FcγRl (CD64), but not to other FcγRs, on effector cells such as macrophages, neutrophils, and dendritic cells. By administering these antibodies to an individual in need thereof, these human or humanized antibodies are capable of treating, ameliorating or slowing the development of the advanced cancer or drug resistant cancer, or a cancer caused or initiated by or sustained by an advanced cancer or drug resistant cancer cell, or a Cancer Stem Cell (CSC). In alternative embodiments, the administered human or humanized antibodies induce an antibody-dependent cell-mediated cytotoxicity (ADCC) reaction against the advanced cancer or drug resistant cancer cell, or CSC.

### RELATED APPLICATIONS

This U.S. utility patent application is a continuation of U.S. application Ser. No. 16/945,057, filed Jul. 31, 2020, which claims the benefit of priority to U.S. Provisional Application No. 62/882,296, filed Aug. 2, 2019. The aforementioned applications are expressly incorporated herein by reference in their entireties and for all purposes.

### STATEMENT AS TO FEDERALLY SPONSORED RESEARCH

This invention was made with government support under CA220512 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Feb. 6, 2026, is named 114198-2318_SL.xml and is 16,872 bytes in size.

### TECHNICAL FIELD

This invention generally relates to immunology and oncology. In alternative embodiments, provided are compositions and methods for treating or ameliorating an advanced cancer such as a drug resistant or a metastatic cancer which express αvβ3 polypeptides on their cell surfaces, or for killing Cancer Stem Cells (CSCs) which express αvβ polypeptides on their cell surfaces, by using human or humanized antibodies capable of specifically binding cell surface-expressed αvβ (avb3) polypeptides whose Fc region has a selective affinity to human FcγRl (CD64), but not to other FcγRs, on effector cells such as macrophages, neutrophils, and dendritic cells. By administering these antibodies to an individual in need thereof, these human or humanized antibodies are capable of treating, ameliorating or slowing the development of the advanced cancer or drug resistant cancer, or a cancer caused or initiated by or sustained by an advanced cancer or drug resistant cancer cell, or a Cancer Stem Cell (CSC). In alternative embodiments, the administered human or humanized antibodies induce an antibody-dependent cell-mediated cytotoxicity (ADCC) reaction against the advanced cancer or drug resistant cancer cell, or CSC.

### BACKGROUND

Antibodies induce antibody-dependent cell-mediated cytotoxicity (ADCC) against target cells utilizing effector cells such as macrophages, natural killer cells, dendritic cells, and neutrophils. To utilize these effector cells, the Fc of antibodies needs to have an affinity to Fcγ receptors (FcγRs) on the effector cells.

### SUMMARY

In alternative embodiments, provided are methods for:
treating or ameliorating cancer, optionally an advanced cancer or a drug resistant cancer, or
killing a Cancer Stem Cells (CSC),

wherein the cancer, the advanced cancer, the drug resistant cancer or the CSC express αvβ polypeptides on their cell surfaces,

comprising administering to an individual in need thereof a human or a humanized antibody capable of Fc region-specific binding to human FcγRl (CD64) receptors but not to, or substantially not to, other human FcγRs, and capable of specifically binding to cell surface-expressed αvβ (avb3) polypeptides,

wherein optionally the human FcγRl (CD64) receptors are expressed on the surface of human macrophages, neutrophils and/or dendritic cells,

thereby inducing an antibody-dependent cell-mediated cytotoxicity (ADCC) response or reaction against the advanced cancer or drug resistant cancer cell, or CSC.

In alternative embodiments of methods as provided herein:
the human or humanized antibody comprises monoclonal antibody (mAb) LM609 (MedImmune), or an mAb having ATCC accession number HB9537, or an mAb as described in U.S. patent serial no. (USSN) 5,753,230;
the human or humanized antibody comprises VITAXIN™ (MedImmune) or MEDI-523;
the human or humanized antibody comprises etaracizumab (or etaratuzumab), or MEDI-522, or ABEGRIN™ (MedImmune);
the method further comprises administration to the individual in need thereof an additional cancer therapeutic agent or therapy, wherein optionally the additional cancer therapeutic agent comprises paclitaxel;
the human or humanized antibody is administered to the individual in need thereof at a dosage of between about 1 to about 8 mg/kg, or between about 0.5 to about 12 mg/kg;
the human or humanized antibody is administered intravenously (IV), intrathecally, sublingually, rectally, intravaginally, subcutaneously or intramuscularly (IM), or is injected or placed in situ near or in approximation to or into the cancer or tumor (optionally a solid tumor), or advanced cancer or a drug resistant cancer, or CSC, or is administered by in situ placement or insertion of an implant comprising the human or humanized antibody;
the additional cancer therapeutic agent or therapy comprises, or is an antibody selected from the group consisting of: abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab and/or any combination thereof,
the additional cancer therapeutic agent or therapy comprises a growth factor inhibitor, wherein optionally the growth factor inhibitor comprises a Receptor Tyrosine Kinase (RTK) inhibitor, a Src inhibitor, an anti-metabolite inhibitor, a gemcitabine, a GEMZAR™, a mitotic poison, a paclitaxel, a taxol, an ABRAXANE™, an erlotinib, a TARCEVA™, a lapatinib, a TYKERB™, a cetuxamib, an ERBITUX™, a PD-1 inhibitor, a PD-L1 inhibitor and/or an insulin growth factor inhibitor; and/or
a plurality of the human or humanized antibodies are pre-incubated ex vivo with the human macrophages, neutrophils, monocytes and/or dendritic cells before administration to the individual in need thereof, wherein optionally the human macrophages, neutrophils, monocytes and/or dendritic cells are activated human macrophages, neutrophils, monocytes and/or dendritic cells, and optionally the dendritic cells or monocytes are activated as set forth in U.S. Pat. No. 10,023,841, and optionally the dendritic cells or monocytes are antigen loaded dendritic cells or monocytes.

In alternative embodiments, provided are uses of a human or humanized antibody capable of Fc region-specific binding to human FcγRl (CD64) receptors but not to, or substantially not to, other human FcγRs, and capable of specifically binding to cell surface-expressed αvβ (avb3) polypeptides, for
treating or ameliorating a cancer or a tumor, or an advanced cancer or a drug resistant cancer, or
killing a Cancer Stem Cell (CSC), wherein the advanced cancer, drug resistant cancer or CSC express αvβ polypeptides on their cell surfaces.

In alternative embodiments, provided are human or humanized antibodies capable of Fc region-specific binding to human FcγRl (CD64) receptors but not to, or substantially not to, other human FcγRs, and capable of specifically binding to cell surface-expressed αvβ (avb3) polypeptides, for use in:
treating or ameliorating a cancer or a tumor, or an advanced cancer or a drug resistant cancer, or
killing Cancer Stem Cells (CSCs),
wherein the advanced cancer, drug resistant cancer or CSC express αvβ polypeptides on their cell surfaces.

The details of one or more exemplary embodiments of the invention are set forth in the description below. Other features, objects, and advantages of the invention will be apparent from the description, figures and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1A-B shows the nucleotide and amino acid sequence of the variable region of the antibody VITAXIN™: FIG. 1A shows the nucleotide and amino acid sequences for the heavy chain variable region (SEQ ID NO:1 and SEQ ID NO:2, respectively) and FIG. 1B shows the nucleotide and amino acid sequences for the light chain variable region (SEQ ID NO:3 and SEQ ID NO:4, respectively).

FIG. 2A-B shows the nucleotide and amino acid sequence of the variable region of the monoclonal antibody LM609; FIG. 2A shows the nucleotide and amino acid sequence of the LM609 heavy chain variable region (SEQ ID NO:5 and SEQ ID NO:6, respectively), the variable region extends from amino acid Glu1 to Ala117; and FIG. 2B shows the nucleotide and amino acid sequence of the LM609 light chain variable region (SEQ ID NO:7 and SEQ ID NO:8, respectively).

FIG. 3 shows a light chain polypeptide (for pairing with an LM609 heavy chain polypeptide variable region amino acid sequence as that shown in FIG. 1A) comprising a variable region amino acid sequence having a nucleotide and amino sequence as set forth in SEQ ID NO:9 and SEQ ID NO:10, respectively, or a functional fragment thereof.

Like reference symbols in the various drawings indicate like elements.

### DETAILED DESCRIPTION

In alternative embodiments, provided are compositions and methods for treating or ameliorating a cancer or a tumor, for example, an advanced cancer such as a drug resistant cancer which express αvβ polypeptides on their cell surfaces, or for killing Cancer Stem Cells (CSCs) which express αvβ polypeptides on their cell surfaces, by using (by administration of) human or humanized antibodies capable of specifically binding cell surface-expressed αvβ (avb3) polypeptides whose Fc region has a selective affinity to human FcγRl (CD64), but not to, or substantially not to, other FcγRs, on effector cells such as macrophages, neutrophils, and dendritic cells. By administering these antibodies to an individual in need thereof, or practicing methods as provided herein, these human or humanized antibodies are capable of treating, ameliorating or slowing the development of the cancer or tumor, or advanced cancer or drug resistant cancer, or the cancer caused or initiated by or sustained by an advanced cancer or drug resistant cancer cell, or a Cancer Stem Cell (CSC). In alternative embodiments, the administered human or humanized antibodies induce an antibody-dependent cell-mediated cytotoxicity (ADCC) reaction against the advanced cancer or drug resistant cancer cell, or CSC.

### Pharmaceutical Compositions and Formulations

In alternative embodiments, provided are pharmaceutical compositions and formulations, e.g., comprising human or humanized antibodies capable of specifically binding cell surface-expressed αvβ (avb3) polypeptides whose Fc region has a selective affinity to human FcγRl (CD64), but not to, or substantially not to, other FcγRs, on effector cells such as macrophages, neutrophils, and dendritic cells, and methods for: treating or ameliorating an advanced cancer such as a drug resistant cancer which express αvβ polypeptides on their cell surfaces, or for killing Cancer Stem Cells (CSCs) which express αvβ polypeptides on their cell surfaces. In alternative embodiments, pharmaceutical compositions and formulations further comprise additional therapeutic agents, or further comprise immune cells such as macrophages, neutrophils, monocytes and dendritic cells, or activated forms thereof, optionally including macrophages, neutrophils, monocytes and dendritic cells that have been pre-incubated ex vivo with the human or humanized antibodies capable of specifically binding cell surface-expressed αvβ (avb3) polypeptides.

In alternative embodiments, compositions provided herein, and compositions used to practice the methods provided herein, are formulated with a pharmaceutically acceptable carrier. In alternative embodiments, the pharmaceutical compositions used to practice the methods provided herein can be administered parenterally, topically, orally, intrathecally, sublingually, rectally, intravaginally, subcutaneously or intramuscularly (IM) or by any form of local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton PA (“Remington's”).

Therapeutic agents as provided herein, e.g., comprising anti-αvβ (avb3) antibodies, and antibodies used to practice methods as provided herein, can be administered alone or as a component of a pharmaceutical formulation (composition), or concurrently with, before and/or after administration with another active agent, e.g., a growth factor inhibitor, wherein optionally the growth factor inhibitor comprises a Receptor Tyrosine Kinase (RTK) inhibitor, a Src inhibitor, an anti-metabolite inhibitor, a gemcitabine, a GEMZAR™, a mitotic poison, a paclitaxel, a taxol, an ABRAXANE™, an erlotinib, a TARCEVA™, a lapatinib, a TYKERB™, a cetuxamib, an ERBITUX™, a PD-1 inhibitor, a PD-L1 inhibitor, or an insulin growth factor inhibitor.

Pharmaceutical compositions and formulations, e.g., comprising anti-αvβ (avb3) antibodies, may be formulated for administration in any convenient way for use in human or veterinary medicine.

Wetting agents, emulsifiers and lubricants, such as sodium lauryl sulfate and magnesium stearate, as well as coloring agents, release agents, coating agents, sweetening, flavoring and perfuming agents, preservatives, buffers and/or antioxidants can also be present in the compositions.

Formulations of the compositions provided herein and as used to practice the methods provided herein include those suitable for oral, nasal, topical, parenteral, rectal, subcutaneous, sublingual, intraocular, intramuscular, intrathecal and/or intravaginal administration.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations provided herein and as used to practice the methods provided herein can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, geltabs, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations provided herein and as used to practice the methods provided herein can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent as provided herein (for example, a human or humanized antibody capable of Fc region-specific binding to human FcγRl (CD64) receptors but not to, or substantially not to, other human FcγRs, and capable of specifically binding to cell surface-expressed αvβ (avb3) polypeptides) antibody, optionally including immune cells) in admixture with excipients suitable for the manufacture of aqueous suspensions. Such excipients include a suspending agent, such as sodium carboxymethylcellulose, methylcellulose, hydroxypropyl-methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration hydrophobic active agents (e.g., an anti-αvβ (avb3) antibody) used to practice the methods provided herein. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations provided herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing embodiment provided herein, the pharmaceutical compounds can also be administered by in intranasal, intravenous (IV), intramuscular, sublingual, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing embodiments provided herein, the pharmaceutical compounds can be delivered by transdermally, by a topical route, formulated as applicator sticks, solutions, suspensions, emulsions, gels, creams, ointments, pastes, jellies, paints, powders, and aerosols.

In practicing embodiments provided herein, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In practicing embodiments provided herein, the pharmaceutical compounds can be parenterally administered, such as by intravenous (IV), intrathecally, sublingually, rectally, intravaginally, subcutaneously administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations provided herein and as used to practice the methods provided herein can be lyophilized. Also provided are stable lyophilized formulations comprising a composition provided herein, which can be made by lyophilizing a solution comprising a pharmaceutical provided herein on and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations provided herein and as used to practice the methods provided herein can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations provided herein and as used to practice the methods provided herein can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). For example, in alternative embodiments, pharmaceutical compositions provided herein are administered in an amount sufficient to: for treating or ameliorating an advanced cancer such as a drug resistant cancer which express αvβ polypeptides on their cell surfaces, or for killing Cancer Stem Cells (CSCs) which express αvβ polypeptides on their cell surfaces. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods provided herein are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation for oral administration of compositions provided herein or as used to practice the methods provided herein can be in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods provided herein can further comprise co-administration with other drugs or pharmaceuticals, e.g., compositions for treating cancer, septic shock, infection, fever, pain and related symptoms or conditions. For example, the methods and/or compositions and formulations provided herein can be co-formulated with and/or co-administered with antibiotics (e.g., antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Antibodies and Antigen Binding Polypeptides as Pharmaceutical Compositions

In alternative embodiments, also provided are compositions and methods comprising antibodies as provided herein, including antibodies used to practice methods as provided herein. In alternative embodiments, provided are compositions to administer these antibodies and polypeptides.

In alternative embodiments, method comprise use of any polypeptide capable of specifically binding cell surface-expressed αvβ (avb3) polypeptides whose Fc region has a selective affinity to human FcγRl (CD64), but not to, or substantially not to, other FcγRs, on effector cells such as macrophages, neutrophils, and dendritic cells.

Alternative embodiments can use “humanized” antibodies, including forms of non-human (e.g., murine) antibodies that are chimeric antibodies comprising minimal sequence (e.g., the antigen binding fragment) derived from non-human immunoglobulin. In alternative embodiments, humanized antibodies are human immunoglobulins in which residues from a hypervariable region (HVR) of a recipient (e.g., a human antibody sequence) are replaced by residues from a hypervariable region (HVR) of a non-human species (donor antibody) such as mouse, rat, rabbit or nonhuman primate having the desired specificity, affinity, and capacity. In alternative embodiments, framework region (FR) residues of the human immunoglobulin are replaced by corresponding non-human residues to improve antigen binding affinity.

In alternative embodiments, humanized antibodies may comprise residues that are not found in the recipient antibody or the donor antibody. These modifications may be made to improve antibody affinity or functional activity. In alternative embodiments, the humanized antibody can comprise substantially all of at least one, and typically two, variable domains, in which all or substantially all of the hypervariable regions correspond to those of a non-human immunoglobulin and all or substantially all of Ab framework regions are those of a human immunoglobulin sequence.

In alternative embodiments, a humanized antibody used to practice embodiments provided herein can comprise at least a portion of an immunoglobulin constant region (Fc), typically that of or derived from a human immunoglobulin.

However, in alternative embodiments, completely human antibodies also can be used to practice embodiments provided herein, including human antibodies comprising amino acid sequence which corresponds to that of an antibody produced by a human. This definition of a human antibody specifically excludes a humanized antibody comprising non-human antigen binding residues.

In alternative embodiments, method comprise use of humanized antibodies capable of specifically binding to an αvβ (avb3) integrin polypeptide, including humanized VITAXIN™ (MedImmune) or MEDI-523, etaracizumab (or etaratuzumab), or MEDI-522, or ABEGRIN™ (MedImmune).

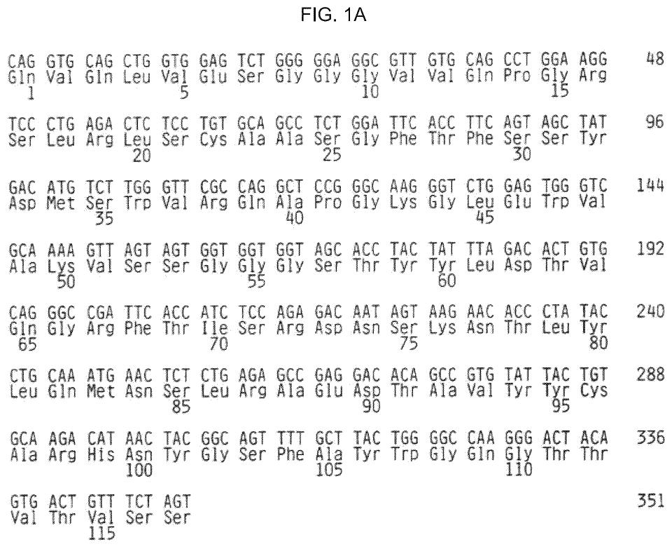

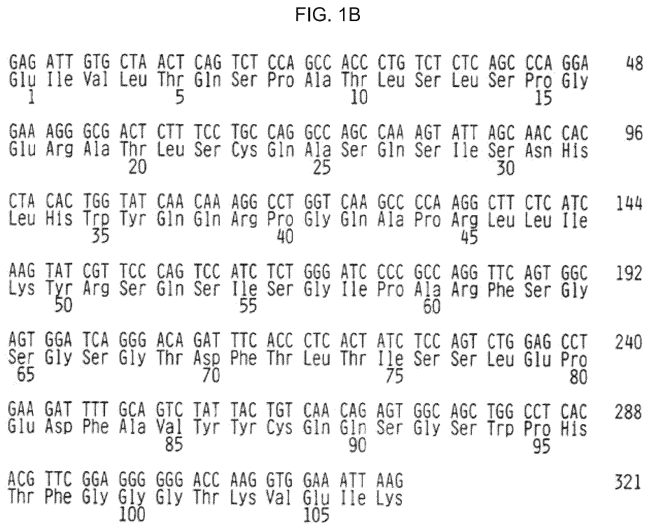

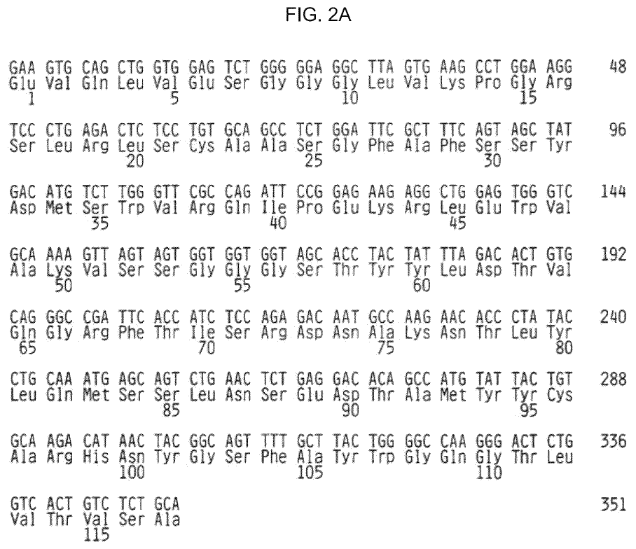

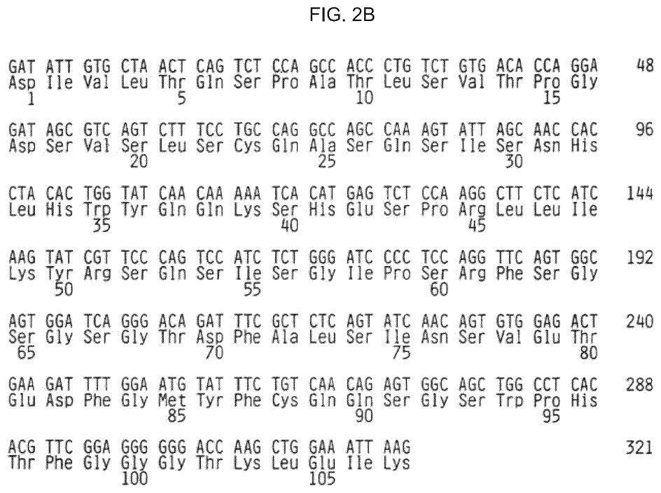

In alternative embodiments, method comprise use of humanized antibodies, for example, VITAXIN™, as described in U.S. Pat. Nos. 6,590,079, 7,422,744 and 7,422,745. In alternative embodiments, an antibody used to practice embodiments as provided herein comprises an antibody exhibiting selective binding affinity to αᵥβ₃ and comprising a heavy chain polypeptide comprising a variable region amino acid sequence as set forth in FIG. 1A (SEQ ID NO:1) and a light chain polypeptide comprising a variable region amino acid sequence as that shown in FIG. 1B (SEQ ID NO:2), or a functional fragments thereof.

In alternative embodiments, antibodies used to practice embodiments provided herein comprise “affinity matured” antibodies, e.g., antibodies comprising with one or more alterations in one or more hypervariable regions which result in an improvement in the affinity of the antibody for antigen; e.g., a histone methyl and/or acetyl transferase, compared to a parent antibody which does not possess those alteration(s). In alternative embodiments, antibodies used to practice embodiments provided herein are matured antibodies having nanomolar or even picomolar affinities for the target antigen, e.g., a histone methyl and/or acetyl transferase. Affinity matured antibodies can be produced by procedures known in the art.

In alternative embodiments, antibodies used to practice methods as provided herein are: the monoclonal antibody LM609; the monoclonal antibody LM609 is described e.g., in Cheresh et al., J Biol Chem. 1987; 262(36):17703-11; and U.S. patent number (USPN) 5,753,230, and U.S. Pat. No. 6,590,079. LM609 is a murine monoclonal antibody specific for the integrin αvβ3, see e.g., Cheresh, D. A., Proc. Natl. Acad. Sci. USA 84:6471-6475 (1987), and Cheresh et al, J. Biol. Chem. 262:17703-17711 (1987). LM609 was produced against and is reactive with the M21 cell adhesion receptor now known as the integrin αvβ3.

LM609 inhibits the attachment of M21 cells to αvβ ligands such as vitronectin, fibrinogen and von Willebrand factor (Cheresh and Spiro, supra) and is also an inhibitor of αvβ3-mediated pathologies such as tumor induced angiogenesis (Brooks et al. Cell 79:1157-1164 (1994)), granulation tissue development in cutaneous wound (Clark et al., Am. J. Pathology, 148:1407-1421 (1996)) and smooth muscle cell migration such as that occurring during restenosis (Choi et al., J. Vascular Surg., 19:125-134 (1994); Jones et al., Proc. Natl. Acad. Sci. 93:2482-2487 (1996)).

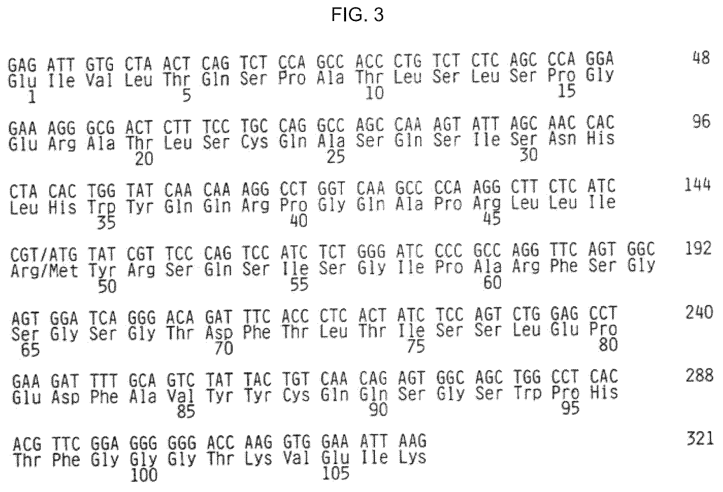

In alternative embodiments, antibodies used to practice methods as provided herein include a grafted LM609 grafted antibody exhibiting selective binding affinity to αᵥβ₃ comprising a heavy chain polypeptide variable region amino acid sequence as that shown in FIG. 1A (SEQ ID NO:2) and a light chain polypeptide comprising substantially a variable region amino acid sequence having a nucleotide and amino sequence as set forth in SEQ ID NO:9 and SEQ ID NO:10 (see FIG. 3), respectively, or a functional fragment thereof.

### Products of Manufacture and Kits

Provided are products of manufacture and kits for practicing methods as provided herein, including comprising human or humanized antibodies capable of specifically binding cell surface-expressed αvβ (avb3) polypeptides whose Fc region has a selective affinity to human FcγRl (CD64), but not to, or substantially not to, other FcγRs; and/or also comprising macrophages, neutrophils, and dendritic cells; and optionally further comprising instructions for practicing methods as provided herein.

Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 110%, 1%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

A number of embodiments of the invention have been described. Nevertheless, it can be understood that various modifications may be made without departing from the spirit and scope of the invention. Accordingly, other embodiments are within the scope of the following claims.

What is claimed is:

1. A method of treating or ameliorating a drug resistant cancer or tumor in a subject suffering therefrom, comprising:
administering an EGFR inhibitor and an antibody comprising a means for binding to avb3 expressed on the surface of a drug resistant cancer cell to the subject, thereby treating or ameliorating a drug resistant cancer or tumor in a subject suffering therefrom.

2. The method of claim 1, wherein the EGFR inhibitor comprises an RTK inhibitor.

3. The method of claim 2, wherein the RTK inhibitor is selected from erlotinib, lapatinib, osimertinib, and cetuximab.

4. The method of claim 2, wherein the RTK inhibitor comprises erlotinib.

5. The method of claim 1, wherein the antibody comprises the complementarity determining regions of LM609, CBL544, ab7166, or ab78289.

6. The method of claim 1, wherein the antibody comprises a heavy chain variable region comprising an amino acid sequence of SEQ ID NOs: 2 or 6 and a light chain variable region comprising an amino acid sequence of SEQ ID NO: 4, 8, or 10.

7. The method of claim 1, wherein the cancer or tumor expresses avb3.

8. The method of claim 1 or 7, wherein the cancer or tumor is an epithelial cell cancer or tumor.

9. The method of claim 1, wherein the EGFR inhibitor is administered prior to the administration of the antibody.

10. The method of claim 9, further comprising detecting the expression of avb3 expression in a sample isolated from the cancer or tumor following administration of the EGFR inhibitor and prior to administration of the antibody.

11. The method of claim 1, wherein the antibody comprises a means for Fc binding to CD64 expressed on a macrophage.

12. The method of claim 1, wherein the cancer or tumor expresses avb3.

13. The method of claim 1, further comprising administering an additional therapeutic agent.

14. The method of claim 13, wherein the additional therapeutic agent comprises a growth factor inhibitor, wherein optionally the growth factor inhibitor comprises a Receptor Tyrosine Kinase (RTK) inhibitor, a Src inhibitor, an anti-metabolite inhibitor, a gemcitabine, a GEMZAR™, amitotiepoison, apaclitaxel, a taxol, an ABRAXANE™, an erlotinib, a TARCEVA™, a lapatinib, a TYKERB™, a cetuxamib, an ERBITUX™, a PD-1 inhibitor, a PD-L1 inhibitor, or an insulin growth factor inhibitor.

15. The method of claim 1, wherein administration is oral, intravenously, intrathecally, subcutaneous, or intramuscularly administration.

16. The method of claim 1, wherein administration is directly into the cancer or tumor.

17. The method of claim 1, wherein the antibody and EGFR inhibitor are administered at least two times.

18. The method of claim 1, wherein the antibody is administered in a pharmaceutical composition further comprising a carrier.

19. The method of claim 1, wherein the antibody is administered after the EGFR inhibitor.

20. The method of claim 1, wherein the antibody is administered before the EGFR inhibitor.
